(19) United States
(12) Patent Application Publication
(10) Pub. No.: US 2016/0216261 A1
(43) Pub. Date: Jul. 28, 2016

(54) SYSTEMS AND METHODS FOR IMPROVING THE ACCURACY OF LATERAL FLOW TESTS USING A FOUR-STRIP CARTRIDGE

(71) Applicant: Polymer Technology Systems, Inc., Indianapolis, IN (US)

(72) Inventor: Jeffrey A. Pierce, Redwood City, CA (US)

(21) Appl. No.: 15/004,570

(22) Filed: Jan. 22, 2016

Related U.S. Application Data
(60) Provisional application No. 62/107,111, filed on Jan. 23, 2015.

(51) Int. Cl. G01N 33/558; G01N 33/72; G01N 33/543

(52) U.S. Cl. CPC G01N 33/558; G01N 33/54393; G01N 33/54386; G01N 33/723; G01N 33/726

(57) ABSTRACT

A system for reducing noise in a lateral flow system includes a cartridge having first, second, third, and fourth lateral flow test strips, the lateral flow test strips providing for a detectable indication of an analyte in the presence of the analyte. The system further includes a meter including a cartridge reading system and a microprocessor, the cartridge reading system configured to detect a signal from each of the first, second, third, and fourth lateral flow test strips, the microprocessor including instructions and configured to average each of the signals.

### CROSS-REFERENCE TO RELATED APPLICATIONS

This application claims the benefit of Provisional Application No. 62/107,111 filed on Jan. 23, 2015, titled “Systems And Methods For Improving The Accuracy Of Lateral Flow Tests Using A Four-Strip Cartridge,” the entire disclosure of which is hereby incorporated by reference.

### BACKGROUND

Lateral flow assay test strips are widely used in a variety of different applications. Many complications exist in the use and reading of lateral flow test strips. One common problem is ensuring an optimal amount of fluid sample flows to the lateral flow test strip. Enough fluid to fully saturate the strip is important; however, if too much fluid flows to a test strip, other parts of the test strip may be flooded, reagents in the test strip may be diluted, and other issues may occur.

Additionally, the production of lateral flow test strips and the addition of reagents typically is imperfect. In testing, “noise” or other imperfections in the test strip process may be read as a signal produced from the analyte being tested for. To combat noise, previously two lateral flow test strips receiving sample from a single sample port have been used.

### BRIEF SUMMARY

In one embodiment, a system for reducing noise in a lateral flow system includes a cartridge having first, second, third, and fourth lateral flow test strips, the lateral flow test strips providing for a detectable indication of an analyte in the presence of the analyte. The system further includes a meter including a cartridge reading system and a microprocessor, the cartridge reading system configured to detect a signal from each of the first, second, third, and fourth lateral flow test strips, the microprocessor including instructions and configured to average each of the signals. Optionally, the first lateral flow test strip is parallel to the second lateral flow strip in the cartridge, and the third lateral flow strip is parallel to the fourth lateral flow strip in the cartridge. Alternatively, there is a narrow gap relative to the size of the first lateral flow test strip, between the first lateral flow test strip and the second lateral flow test strip. In one alternative, a barrier is located in the narrow gap. In another alternative, a noise reduction for the system is greater than 20% as compared to a cartridge having two lateral flow test strips. Optionally, the analyte is A1C.

In one embodiment, a method of reducing noise in a lateral flow system includes providing a cartridge having first, second, third, and fourth lateral flow test strips, the lateral flow test strips providing for a detectable indication of an analyte in the presence of the analyte. The method further includes placing a sample in the cartridge to be tested and reading the cartridge with a meter. The method further includes calculating an average of a signal coming from each of the first, second, third, and fourth lateral flow test strips, the signal representing an amount of the analyte. Optionally, the first lateral flow test strip is parallel to the second lateral flow strip in the cartridge, and the third lateral flow strip is parallel to the fourth lateral flow strip in the cartridge. Alternatively, there is a narrow gap, relative to the size of the first lateral flow test strip, between the first lateral flow test strip and the second lateral flow test strip. In one alternative, a barrier is located in the narrow gap. In another alternative, a noise reduction for the method is greater than 20% as compared to a cartridge having two lateral flow test strips. Alternatively, the analyte is A1C.

In one embodiment, a cartridge for reducing noise in a lateral flow system includes first, second, third, and fourth lateral flow test strips, the lateral flow test strips providing for a detectable indication of an analyte in the presence of the analyte, wherein the cartridge is readable by a meter, and the meter includes instructions and is configured to detect a signal from each of the first, second, third, and fourth lateral flow test strips, and to average each of the signals. Optionally, the first lateral flow test strip is parallel to the second lateral flow strip in the cartridge, and the third lateral flow strip is parallel to the fourth lateral flow strip in the cartridge. Alternatively, there is a narrow gap relative to the size of the first lateral flow test strip between the first lateral flow test strip and the second lateral flow test strip. Optionally, a barrier is located in the narrow gap. In one alternative, the analyte is A1C.

### BRIEF DESCRIPTION OF THE FIGURES

FIG. 1 shows one embodiment of a four-strip cartridge;

FIG. 2 is a graph of the actual imprecision percentage compared to the imprecision for averaged strips having two lateral flow strips;

FIG. 3 is a hypothetical graph, based on the actual data from the graph of FIG. 2, for a system that averages four test strips.

### DETAILED DESCRIPTION

Certain terminology is used herein for convenience only and is not to be taken as a limitation on the embodiments of the systems and methods for improving the accuracy of lateral flow tests using a four-strip cartridge. In the drawings, the same reference letters are employed for designating the same elements throughout the several figures.

In order decrease the noise and increase the signal in a cartridge system having lateral flow, in one embodiment, additional lateral flow test strips are added. The signal from all of the strips then is averaged. This is a type of random strip averaging. In an alternative embodiment, instead of automatically averaging all four strips, the meter includes an algorithm for disregarding one or more signals from the lateral flow test strips. Signals may be disregarded for a number of reasons. In one configuration, the system may perform multiple averages, in each scenario leaving one of the signals from one of the strips out. The system then may compare the left-out signal to the average of the other signals. If the left-out signal is more than two standard deviations (alternatively, a different distance may be used) from the average of the other signals, based on the historical precision of the device, then that left-out signal is disregarded. If none of the signals are a significant distance from the average of the others, then all of the signals may be averaged and used. If all of the signals are a significant distance from the average of the other signals, then the device may either average all of the signals or issue an error code that the test was a failure.

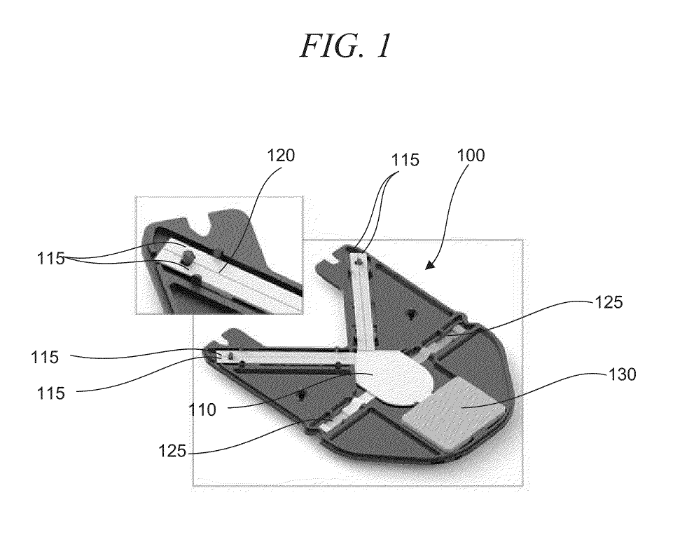

In many configurations, the previous number of two lateral flow test strips is increased to four. This may be done by reducing the width of the lateral flow test strips in some embodiments and essentially splitting each lateral flow strip into two strips having more narrow widths. FIG. 1 shows one embodiment of a four-strip cartridge 100. This cartridge is similar to previous two-strip cartridges. Four-strip cartridge 100 includes a sample pad 110 that is configured to receive a fluid sample (in many cases blood, but other bodily fluids may be utilized as well). Four-strip cartridge 100 includes four lateral flow test strips 115. The lateral flow test strips 115 on either side of the device are separated by a thin gap 120. In alternative embodiments, a plastic shield may be placed in thin gap 120, or the molded body of the four-strip cartridge 100 may include a plastic shield as part of the body structure. The four-strip cartridge 100 further includes autostart leads 125 that absorb fluid when a sample is provided and provide an optical indication to the meter that testing has started. These autostart leads 125 may be used to automatically start the meter. Additionally included is an excess-sample absorbent pad 130, which assists in controlling the available sample amount by absorbing sample above a certain height that is pooled on sample pad 110.

Alternative embodiments are available, whereby more completely separated additional strips are included. These lateral flow strips may be positioned in a parallel position to existing strip locations, or they may be set at an angle to existing strip locations.

By doubling the test cartridge strip count from two to four to average out more strip-to-strip noise, based on random noise, it is expected that there would be about 30% precision improvement.

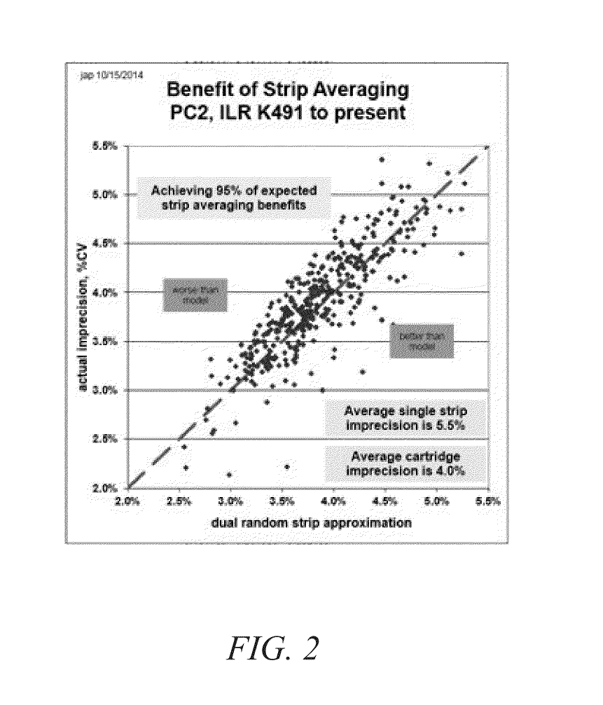

FIG. 2 is a graph of the actual imprecision percentage compared to the imprecision for averaged strips having two lateral flow strips. There is a clear benefit of random strip averaging. The average single strip imprecision is 5.5%, while the average cartridge imprecision is only 4.0%.

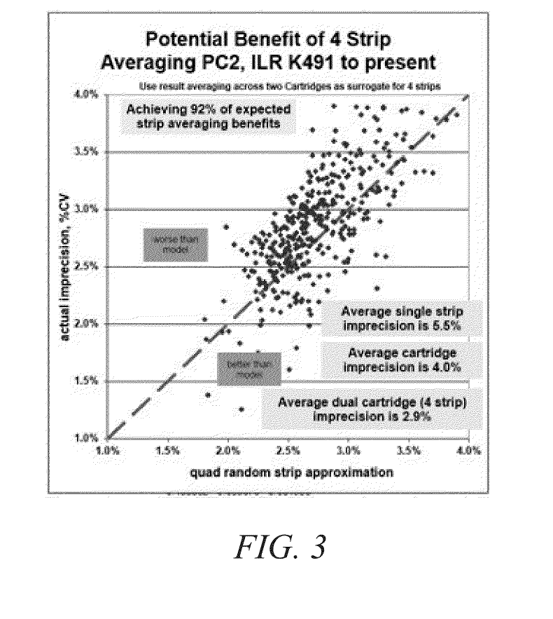

FIG. 3 shows the same data set used to examine the averaging of two Test Cartridge results together (used average of two adjacent cartridge results when sorted in build/bag order) as a surrogate for the additional averaging benefit that might be possible if four strips were available for averaging rather than two. The additional benefit shown below is again rather close to that predicted from a purely mathematical exercise of averaging results from random strips. The average dual cartridge four-strip imprecision is reduced to 2.9% under this hypothetical model.

In many embodiments, the cartridge may provide testing for A1C or other analytes that may be found in the blood.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof. Note that, although particular embodiments are shown, features of each attachment may be interchanged between embodiments.

What is claimed as new and desired to be protected by Letters Patent of the United States is:

1. A system for reducing noise in a lateral flow system, comprising:
a cartridge having first, second, third, and fourth lateral flow test strips, the lateral flow test strips providing for a detectable indication of an analyte in the presence of the analyte; and
a meter including a cartridge reading system and a microprocessor, the cartridge reading system configured to detect a signal from each of the first, second, third, and fourth lateral flow test strips, the microprocessor including instructions and configured to average each of the signals.

2. The system of claim 1, wherein the first lateral flow test strip is parallel to the second lateral flow strip in the cartridge, and the third lateral flow strip is parallel to the fourth lateral flow strip in the cartridge.

3. The system of claim 2, wherein there is a narrow gap relative to the size of the first lateral flow test strip between the first lateral flow test strip and the second lateral flow test strip.

4. The system of claim 3, wherein a barrier is located in the narrow gap.

5. The system of claim 1, wherein a noise reduction for the system is greater than 20% as compared to a cartridge having two lateral flow test strips.

6. The system of claim 1, wherein the analyte is A1C.

7. A method of reducing noise in a lateral flow system, comprising:
providing a cartridge having first, second, third, and fourth lateral flow test strips, the lateral flow test strips providing for a detectable indication of an analyte in the presence of the analyte;
placing a sample in the cartridge to be tested;
reading the cartridge with a meter; and
calculating an average of a signal coming from each of the first, second, third, and fourth lateral flow test strips, the signal representing an amount of the analyte.

8. The method of claim 7, wherein the first lateral flow test strip is parallel to the second lateral flow strip in the cartridge, and the third lateral flow strip is parallel to the fourth lateral flow strip in the cartridge.

9. The method of claim 7, wherein there is a narrow gap relative to the size of the first lateral flow test strip between the first lateral flow test strip and the second lateral flow test strip.

10. The method of claim 9, wherein a barrier is located in the narrow gap.

11. The method of claim 7, wherein a noise reduction for the method is greater than 20% as compared to a cartridge having two lateral flow test strips.

12. The method of claim 7, wherein the analyte is A1C.

13. A cartridge for reducing noise in a lateral flow system, the cartridge comprising:
first, second, third, and fourth lateral flow test strips, the lateral flow test strips providing for a detectable indication of an analyte in the presence of the analyte, wherein the cartridge is readable by a meter and the meter includes instructions and is configured to detect a signal from each of the first, second, third, and fourth lateral flow test strips and to average each of the signals.

14. The cartridge of claim 13, wherein the first lateral flow test strip is parallel to the second lateral flow strip in the cartridge, and the third lateral flow strip is parallel to the fourth lateral flow strip in the cartridge.

15. The cartridge of claim 14, wherein there is a narrow gap relative to the size of the first lateral flow test strip between the first lateral flow test strip and the second lateral flow test strip.

16. The cartridge of claim 15, wherein a barrier is located in the narrow gap.

17. The cartridge of claim 13, wherein the analyte is A1C.
